(19) United States
(12) Patent Application Publication
(10) Pub. No.: US 2025/0302724 A1
(43) Pub. Date: Oct. 2, 2025

(54) CHITOSAN-BIOTIN COMPOSITION, DEVICE, AND METHOD

(71) Applicant: Amita Vadada, Jersey City, NJ (US)

(72) Inventor: Amita Vadada, Jersey City, NJ (US)

(21) Appl. No.: 19/096,839

(22) Filed: Apr. 1, 2025

Related U.S. Application Data
(60) Provisional application No. 63/572,435, filed on Apr. 1, 2024.

(51) Int. Cl. A61K 8/73; A61K 8/67; A61Q 7/00

(52) U.S. Cl. CPC A61K 8/736; A61K 8/673; A61Q 7/00; A61K 2800/74

(57) ABSTRACT

A method for promoting eyelash growth may include applying an effective amount of a chitosan-biotin composition to the eyelashes and preferably to the eyelash line of a patient or recipient to promote the growth and enhance the appearance of the eyelashes of a mammalian subject, such as a human user, patient, recipient, etc. The chitosan-biotin composition may include a chitosan biotin polymer having approximately a 1:1 to 1:10 chitosan to biotin ratio, in which the chitosan-biotin composition may include approximately between 0.001% to 0.1% of the chitosan biotin polymer.

### CROSS REFERENCE TO RELATED APPLICATIONS

This application claims priority to and the benefit of the filing date of U.S. Provisional Application No. 63/572,435, filed on Apr. 1, 2024, entitled “CHITOSAN-BIOTIN COMPOSITION, DEVICE, AND METHOD”, which is hereby incorporated by reference in its entirety.

### FIELD OF THE INVENTION

This patent specification relates to the field of compositions, devices and methods for promoting eyelash growth. More specifically, this patent specification relates to compositions, devices and methods that include chitosan-biotin for promoting eyelash growth.

### BACKGROUND

Current eyelash enhancing serums often use prostaglandins to grow eyelashes. Ophthalmologists often prescribe prostaglandins for medical treatment of glaucoma. Prostaglandins are efficacious medications widely used as first line treatment for glaucoma. However, patients using prostaglandins have well-recognized cosmetic side effects, and the term “Prostaglandin-Associated Periorbitopathy Syndrome” (PAPS) was coined to refer to the constellation of side effects from prostaglandin use. Features of PAPS include upper eyelid ptosis, periorbital fat atrophy, deepening of the upper eyelid sulcus, inferior scleral show, and hyperpigmentation of the periorbital skin. Prostaglandin use is also associated with periocular erythema, blepharitis, and darkening of the iris. These changes may be partially or fully reversible upon discontinuation of the prostaglandin.

Therefore, a need exists for novel compositions, devices and methods for promoting the growth and enhancing the appearance of eyelashes in a healthy way, without using prostaglandins. A further need exists for novel devices and methods which provide targeted and concentrated delivery of the cosmetic product directly to the eyelash root.

### BRIEF SUMMARY OF THE INVENTION

According to one aspect consistent with the principles of the invention, a chitosan-biotin composition for promoting eyelash growth is provided. The chitosan-biotin composition may be used for stimulating the growth and enhancing the appearance of eyelashes. In some embodiments, the composition may include a chitosan biotin polymer having approximately a 1:1 to 1:10 chitosan to biotin ratio, and the chitosan-biotin composition may have approximately between 0.001% to 0.1% chitosan biotin polymer.

According to another aspect consistent with the principles of the invention, a chitosan-biotin composition application device is provided which may be used to apply chitosan-biotin composition to the eyelash and eyelash line to promote the growth and enhance the appearance of eyelashes. In some embodiments, the device may include: a reservoir body having a reservoir cavity; a chitosan-biotin composition having approximately a 1:1 to 1:10 chitosan to biotin ratio, and the chitosan-biotin composition may have approximately between 0.001% to 0.1% chitosan biotin polymer may be contained in the reservoir cavity; and an applicator may be configured to withdraw chitosan-biotin composition and to apply the chitosan-biotin composition to the eyelashes and preferably to the eyelash line of a subject. The applicator preferably may include a brush having a plurality of soft natural and/or synthetic bristles that may be suitable for contacting eyelashes and eyelash line of a subject.

According to another aspect consistent with the principles of the invention, a method for promoting eyelash growth is provided. The method may be used to promote the growth and enhance the appearance of eyelashes of a mammalian subject, such as a human. In some embodiments, the method may include the steps of: applying an effective amount of chitosan-biotin composition to the eyelashes, and optionally to the eyelash line of the mammalian subject. The chitosan-biotin composition may have approximately a 1:1 to 1:10 chitosan to biotin ratio, and the chitosan-biotin composition may include approximately between 0.001% to 0.1% chitosan biotin polymer. Preferably, the method may include re-applying an effective amount of the chitosan-biotin composition to the eyelashes, and optionally to the eyelash line of the mammalian subject after a period of time has elapsed, such as once a day.

### BRIEF DESCRIPTION OF THE DRAWINGS

Some embodiments of the present invention are illustrated as an example and are not limited by the figures of the accompanying drawings, in which like references may indicate similar elements and in which:

FIG. 1—FIG. 1 depicts an example of how chitosan may be biotinylated through an amide bond between the amine of chitosan and the carboxylic acid of biotin to form a chitosan biotin polymer according to various embodiments described herein.

FIG. 2—FIG. 2 illustrates an example chitosan biotin polymer according to various embodiments described herein.

FIG. 3—FIG. 3 shows a perspective view of an example of a chitosan-biotin composition application device according to various embodiments described herein.

FIG. 4—FIG. 4 depicts a sectional elevation view of an example of a reservoir body of a chitosan-biotin composition application device having chitosan-biotin composition in its reservoir cavity according to various embodiments described herein.

FIG. 5—FIG. 5 illustrates a block diagram of an example of a method for promoting eyelash growth, such as which may be used to promote the growth and enhance the appearance of eyelashes, according to various embodiments described herein.

### DETAILED DESCRIPTION OF THE INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

For purposes of description herein, the terms “upper,” “lower,” “left,” “right,” “rear,” “front,” “side,” “vertical,” “horizontal,” and derivatives thereof shall relate to the invention as oriented in FIG. 1. However, one will understand that the invention may assume various alternative orientations and step sequences, except where expressly specified to the contrary. Therefore, the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

Although the terms “first,” “second,” etc. are used herein to describe various elements, these elements should not be limited by these terms. These terms are only used to distinguish one element from another element. For example, the first element may be designated as the second element, and the second element may be likewise designated as the first element without departing from the scope of the invention.

As used in this application, the term “about” or “approximately” refers to a range of values within plus or minus 20% of the specified number. Additionally, as used in this application, the term “substantially” means that the actual value is within about 10% of the actual desired value, particularly within about 5% of the actual desired value and especially within about 1% of the actual desired value of any variable, element or limit set forth herein.

A new chitosan-biotin composition, device, and method for promoting eyelash growth are discussed herein. In the following description, for purposes of explanation, numerous specific details are set forth in order to provide a thorough understanding of the present invention. It will be evident, however, to one skilled in the art that the present invention may be practiced without these specific details.

The present disclosure is to be considered as an exemplification of the invention and is not intended to limit the invention to the specific embodiments illustrated by the figures or description below.

The present invention will now be described by example and through referencing the appended figures representing preferred and alternative embodiments. According to one aspect consistent with the principles of the invention, a chitosan-biotin composition 100 is provided which may be used to promote the growth and/or to enhance the appearance of eyelashes of a mammalian subject, such as a human subject. Human lashes grow in up to six distinct layers on each eyelash line on the upper and lower eyelids, in some cases resulting in sets of over 200 lashes surrounding each eye.

Chitosan is a derivative of chitin, one of the most abundant renewable resources on Earth. Chitin and its derivative chitosan are very abundant polymers in nature, second only to cellulose. Chitosan is 100% biocompatible, biodegradable, and hypoallergenic. It is the only naturally occurring biopolymer that has a positive electrostatic charge. The cationic nature of chitosan gives it a strong affinity to many anions and molecules.

Chitin is a major component of the cell walls of fungi, insect exoskeletons, and crustacean shells. Referring to FIG. 1, chitosan is a natural polycationic polysaccharide derived from partial deacetylation of chitin. In preferred embodiments, the chitosan-biotin composition may comprise chitosan that is sourced from a vegan mushroom source.

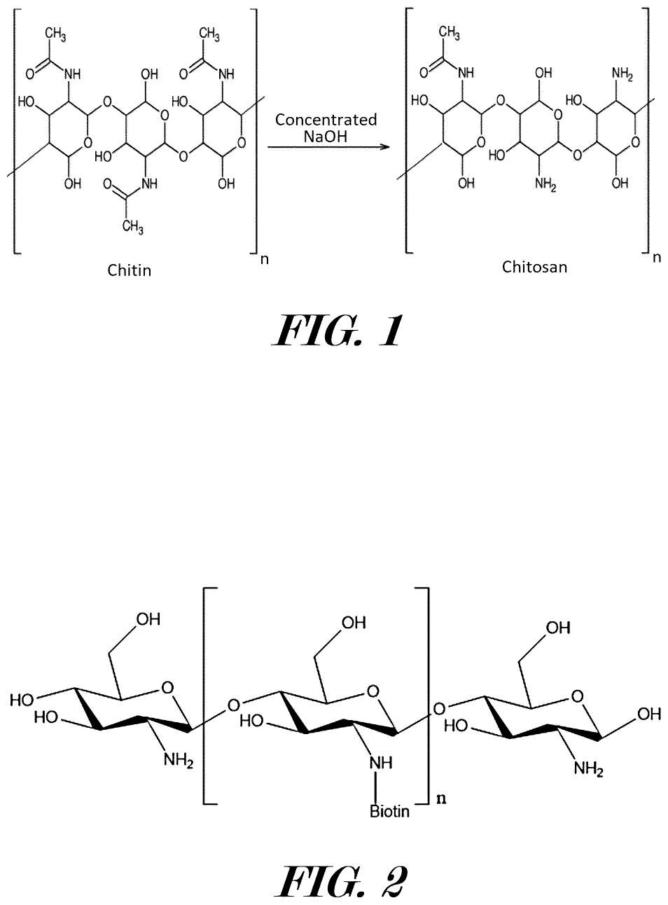

The chitosan-biotin composition 100 comprises a chitosan biotin polymer. Referring to FIG. 2, an example of a chitosan biotin polymer is shown. Chitosan is biotinylated through an amide bond between the amine of chitosan and the carboxylic acid of biotin to form a chitosan biotin polymer (biotinylated chitosan). A chitosan-biotin polymer is a biofunctionalized polymer where the natural polysaccharide chitosan is chemically modified by attaching one or more biotin molecules, enhancing its properties for biomedical applications like drug delivery and targeted therapies.

In some embodiments, the chitosan-biotin composition 100 may comprise a chitosan biotin polymer having a chitosan to biotin ratio of between 1:1 to 1:10. In further embodiments, the chitosan to biotin ratio of the chitosan biotin polymer may be between 1:1 to 1:5. In further embodiments, the chitosan to biotin ratio of the chitosan biotin polymer may be between 1:1 to 1:4. In further embodiments, the chitosan to biotin ratio of the chitosan biotin polymer may be between 1:1 to 1:3. In more preferred embodiments, the chitosan-biotin composition 100 may comprise a chitosan biotin polymer having approximately a 1:2 chitosan to biotin ratio (e.g., every polymer of chitosan may include two molecules of biotin, such that for every polymer of chitosan delivered to an eyelash, two molecules of biotin would be delivered to the eyelash).

In some embodiments, the chitosan-biotin composition 100 may comprise approximately between 0.001% to 0.1% weight per volume (w/v) of the chitosan biotin polymer. In preferred embodiments, the chitosan-biotin composition 100 may comprise approximately between 0.001% to 0.05% of the chitosan biotin polymer. In further embodiments, the chitosan-biotin composition 100 may comprise approximately between 0.002% to 0.01% of the chitosan biotin polymer. In further embodiments, the chitosan-biotin composition 100 may comprise approximately between 0.005% to 0.007% of the chitosan biotin polymer.

There are different molecular weights of chitosan. The molecular weight of chitosan can range between <3 to 2000 kilodalton (kDa). Based on its molecular weight, chitosan can be categorized into very low molecular weight (oligosaccharides weighing<3 kDa), low molecular weight (3-100 kDa), medium molecular weight (100 kDa-1000 kDa), and high molecular weight (>1000 kDa). In preferred embodiments, the chitosan biotin polymer of the chitosan-biotin composition 100 may comprise chitosan having a low or medium-size molecular weight, such as 50 to 250 kDa. In further preferred embodiments, the chitosan biotin polymer of the chitosan-biotin composition 100 may comprise chitosan having a molecular weight of approximately 250 kDa, and more preferred the chitosan biotin polymer of the chitosan-biotin composition 100 may comprise chitosan having a molecular weight of 250 kDa. In further embodiments, the chitosan biotin polymer of the chitosan-biotin composition 100 may comprise chitosan of any other molecular weight.

Chitin is deacetylated to form chitosan, and the resulting amino group on chitosan gives it its positive charge; it appears to be the only polycation in nature. The cationic nature of chitosan allows it to adhere to the negatively charged keratin found in eyelash roots, eyelashes and hair. The attached biotin in a Chitosan-Biotin polymer is thereby delivered directly to the eyelashes and eyelash roots via this electrostatic attraction. Chitosan's cationic nature also gives it a mucoadhesive property, allowing increased bioavailability of biotin at the eyelash root.

In some embodiments, the chitosan-biotin composition 100 may be a topical composition in a form that may include suspensions, oils, creams, ointments, gels, and lotions.

In some embodiments, the chitosan-biotin composition 100 may be a topical composition further comprising a carrier that the chitosan biotin polymer may be integrated with. In preferred embodiments, one or more carriers that the chitosan biotin polymer may be integrated with may include castor oil, Medium-Chain Triglyceride oil (MCT) oil, Glycerin, and Panthenol. In further embodiments, one or more carriers that the chitosan biotin polymer may be integrated with may include water, alcohols, aloe vera gel, allantoin, glycerin, vitamin A and E oils, mineral oil, propylene glycol, dimethyl isosorbide, polypropylene glycol, and 2 myristyl propionate. In further embodiments, the chitosan-biotin composition 100 may be a topical composition further comprising one or more of: Apple stem cell fruit culture, Xanthan gum, Lecithin, Myristoyl Pentapeptide-17, Myristoyl Hexapeptide-16, Tocopheryl acetate, Hyaluronic acid, Jojoba oil, Pomegranate seed oil, Rosemary leaf extract, Caffeine, Maqui berry fruit extract, Gluconolactone, Lutein extract, Turmeric root extract, Sodium benzoate, and Calcium gluconate.

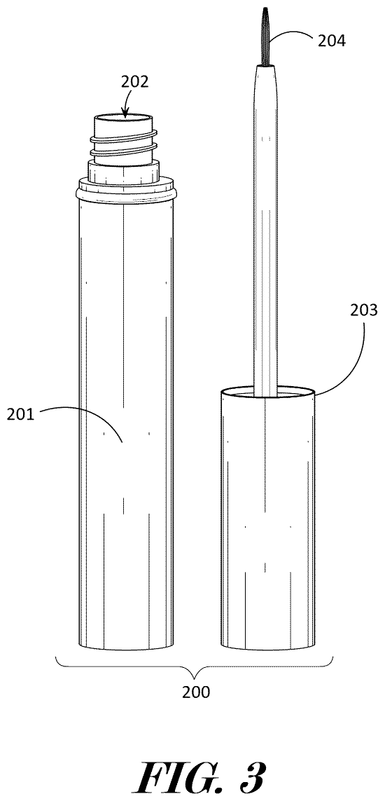

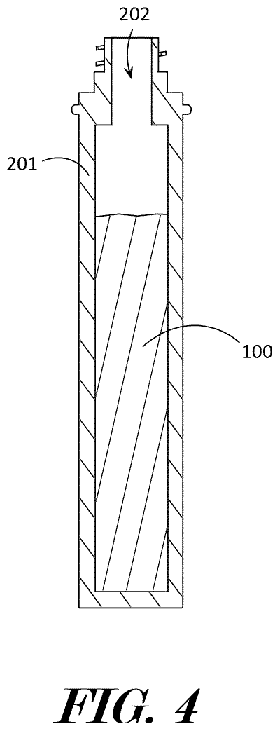

According to another aspect consistent with the principles of the invention, a chitosan-biotin composition application device (“the device”) 200 (FIGS. 3 and 4) is provided which may be used to apply chitosan-biotin composition 100 to the eyelashes and eyelash line to promote the growth and enhance the appearance of eyelashes. In preferred embodiments, the device 200 may comprise a reservoir body 201 having a reservoir cavity 202, and the chitosan-biotin composition 100 may be in the form of a liquid, gel, etc., that may be contained in the reservoir cavity 202. The device 200 may include an applicator 203 which may be configured to withdraw chitosan-biotin composition 100 and to apply the chitosan-biotin composition 100 to the eyelash and eyelash line of a subject. Preferably, the applicator 203 may include a brush 204 having a plurality of soft natural and/or synthetic bristles that may be suitable for contacting eyelashes and eyelash line of a subject. In other embodiments, an applicator 203 may include a sponge, capillary tube, or any other device or method that may be suitable for applying a composition to the eyelashes and eyelash line of a user.

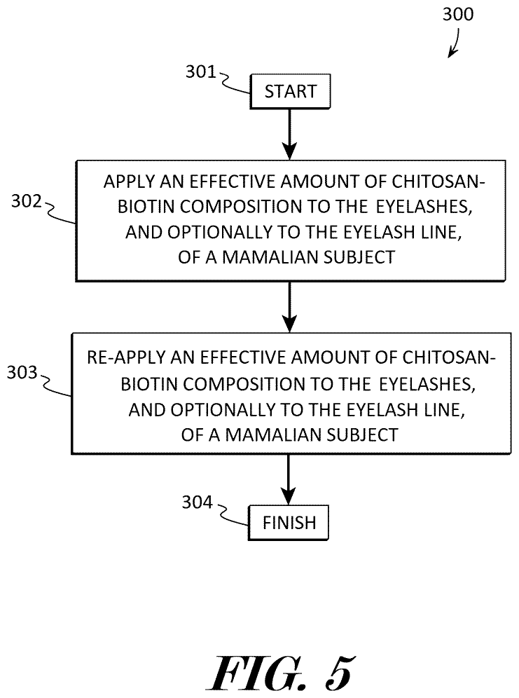

According to another aspect consistent with the principles of the invention, a method for promoting eyelash growth (“the method”) 300 is provided (FIG. 5). The method 300 may be used to promote the growth and enhance the appearance of eyelashes in a mammalian subject. In preferred embodiments, the method 300 may include applying a chitosan-biotin composition 100, as described above, to the eyelashes and preferably to the eyelash line to promote the growth and enhance the appearance of eyelashes of a mammalian subject.

In some embodiments, the method 300 may start 301 and an effective amount of the chitosan-biotin composition 100 may be applied to the eyelashes of a mammalian subject, such as a human user, patient, or recipient, etc., in step 302. In preferred embodiments, the chitosan-biotin composition 100 may be applied to the eyelashes and to the eyelash line eyelash line of a human subject. The phrase “eyelash line” is intended to refer to the edge of eyelid from which eyelash hairs protrude.

An effective amount of the chitosan-biotin composition 100 applied in step 302 may comprise a volume of the chitosan-biotin composition 100 that is sufficient to contact at least 5% of the lower length (portion proximate to the eyelash line) of one or more, and more preferably all, of the eyelashes associated with the eye of a mammalian subject. In preferred embodiments, an effective amount of the chitosan-biotin composition 100 applied in step 302 may comprise a volume of the chitosan-biotin composition 100 that is sufficient to contact at least the lower 10% of one or more, and more preferably all, of the eyelashes and to contact the eyelash line associated with the eye of a mammalian subject. Typically, an effective amount of the chitosan-biotin composition 100 applied at one time in step 302 may comprise between 2 microliters (μL) to 20 microliters (μL). Preferably, a typical volume of chitosan-biotin composition 100 per swipe for a small brush (eyelash application brush) is 2-3 microliters (μL) per swipe so an effective amount of the chitosan-biotin composition 100 applied at one time in step 302 may comprise a user selected amount to cover all or a portion of a desired number of eyelashes and/or to cover a desired amount of one or both eyelash lines of the subject.

In some embodiments, the chitosan-biotin composition 100 may comprise a chitosan biotin polymer having a chitosan to biotin ratio of between 1:1 to 1:10. In further embodiments, the chitosan to biotin ratio of the chitosan biotin polymer may be between 1:1 to 1:5. In further embodiments, the chitosan to biotin ratio of the chitosan biotin polymer may be between 1:1 to 1:4. In further embodiments, the chitosan to biotin ratio of the chitosan biotin polymer may be between 1:1 to 1:3. In more preferred embodiments, the chitosan-biotin composition 100 may comprise a chitosan biotin polymer having approximately a 1:2 chitosan to biotin ratio (e.g., every polymer of chitosan may include two molecules of biotin, such that for every polymer of chitosan delivered to an eyelash, two molecules of biotin would be delivered to the eyelash).

In some embodiments, the chitosan-biotin composition 100 may comprise approximately between 0.001% to 0.1% of the chitosan biotin polymer. In preferred embodiments, the chitosan-biotin composition 100 may comprise approximately between 0.001% to 0.05% of the chitosan biotin polymer. In further embodiments, the chitosan-biotin composition 100 may comprise approximately between 0.002% to 0.01% of the chitosan biotin polymer. In further embodiments, the chitosan-biotin composition 100 may comprise approximately between 0.005% to 0.009% of the chitosan biotin polymer, such as 0.006% of the chitosan biotin polymer.

In preferred embodiments, the chitosan biotin polymer of the chitosan-biotin composition 100 may comprise chitosan having a low or medium-size molecular weight, such as 50 to 250 kDa. In further preferred embodiments, the chitosan biotin polymer of the chitosan-biotin composition 100 may comprise chitosan having a molecular weight of approximately 250 kDa, and more preferred the chitosan biotin polymer of the chitosan-biotin composition 100 may comprise chitosan having a molecular weight of 250 kDa. In further embodiments, the chitosan biotin polymer of the chitosan-biotin composition 100 may comprise chitosan of any other molecular weight.

In some embodiments, the method 300 may use a chitosan-biotin composition application device 200 which may be used to apply chitosan-biotin composition 100 to the eyelashes and eyelash line to promote the growth and enhance the appearance of eyelashes. In preferred embodiments, the device 200 may comprise a reservoir body 201 having a reservoir cavity 202, and the chitosan-biotin composition 100 may be in the form of a liquid, gel, etc., that may be contained in the reservoir cavity 202. The device 200 may include an applicator 203 which may be configured to withdraw chitosan-biotin composition 100 and to apply the chitosan-biotin composition 100 to the eyelashes and eyelash line of a patient or recipient, such as the user. Preferably, the applicator 203 may include a brush 204 having a plurality of soft natural and/or synthetic bristles that may be suitable for contacting and applying the chitosan-biotin composition 100 to the eyelashes and eyelash line.

In step 303, the user may re-apply an effective amount of chitosan-biotin composition 100, as detailed in step 302, to the eyelashes, and optionally to the eyelash line of the mammalian subject. In some embodiments, the user may re-apply an effective amount of chitosan-biotin composition 100 after a period of approximately 24 hours has passed from the initial application in step 302, so that the effective amount of chitosan-biotin composition 100 may be reapplied at least once a day, such as once nightly. In further embodiments, the user may re-apply an effective amount of chitosan-biotin composition 100 after a period of approximately 12 hours has passed from the initial application in step 302, so that the effective amount of chitosan-biotin composition 100 may be reapplied twice a day. In further embodiments, the user may re-apply an effective amount of chitosan-biotin composition 100 after a user desired time period has passed from the initial application in step 302, so that the effective amount of chitosan-biotin composition 100 may be reapplied on an ‘as needed’ or “pro re nata” basis.

In some embodiments, the user may re-apply an effective amount of chitosan-biotin composition 100 over a treatment period of one week, two weeks, four weeks, two months, four months, six months, one year, two years, or any other time period that may be desired. Preferably, the chitosan-biotin composition 100 may be applied repeatedly for a sustained period of time topically on the part of the body to be treated, for example, the eyelids and eyelashes. The preferred dosage regimen will generally involve regular, such as daily, administration for a period of treatment of at least one month and more preferably the period of treatment may be continuous to maintain and promote eyelash growth. Once the desired treatment period has been performed, the method 300 may optionally finish 304 or preferably the period of treatment may proceed indefinitely.

The following treatment examples in which the chitosan-biotin composition 100 and method promoted eyelash growth in human subjects are submitted to illustrate but not to limit this invention. All subjects reported promoted eyelash growth and that their eyelashes overall looked healthier.

### Treatment Example 1

A 34 year old female applied approximately 4 to 6 microliters (μL) of the chitosan-biotin composition 100 having 0.006% chitosan biotin polymer, the chitosan biotin polymer having a 1:2 Chitosan:biotin ratio, to her upper eyelashes and eyelids at the eyelash line every night for 8 weeks, and the female subject reported promoted or improved eyelash growth that included a 25% increase in eyelash length, 50% increase in lash thickness, and 50% increase in eyelash strength.

### Treatment Example 2

A 28 year old female applied approximately 4 to 6 microliters (μL) of the chitosan-biotin composition 100 having 0.005% chitosan biotin polymer, the chitosan biotin polymer having a 1:2 Chitosan:biotin ratio, to her upper eyelashes and eyelids at the eyelash line every night for 8 weeks, and the female subject reported promoted or improved eyelash growth that included a 30% increase in eyelash length, 40% increase in lash thickness, and 50% increase in eyelash strength.

### Treatment Example 3

A 43 year old female applied approximately 4 to 6 microliters (μL) of the chitosan-biotin composition 100 having 0.008% chitosan biotin polymer, the chitosan biotin polymer having a 1:2 Chitosan:biotin ratio, to her upper eyelashes and eyelids at the eyelash line every night for 8 weeks, and the female subject reported promoted or improved eyelash growth that included a 20% increase in eyelash length, 40% increase in lash thickness, and 50% increase in eyelash strength.

Additionally, while some materials have been provided, in other embodiments, the elements that comprise the device 200 may be made from or may comprise durable materials such as aluminum, steel, other metals and metal alloys, wood, hard rubbers, hard plastics, fiber reinforced plastics, carbon fiber, fiberglass, resins, polymers or any other suitable materials including combinations of materials. Additionally, one or more elements may be made from or may comprise durable and slightly flexible materials such as soft plastics, silicone, soft rubbers, or any other suitable materials including combinations of materials. In some embodiments, one or more of the elements that comprise the device 200 may be coupled or connected together with heat bonding, chemical bonding, adhesives, clasp type fasteners, clip type fasteners, rivet type fasteners, threaded type fasteners, other types of fasteners, or any other suitable joining method. In other embodiments, one or more of the elements that comprise the device 200 may be coupled or removably connected by being press fit or snap fit together, by one or more fasteners such as hook and loop type or Velcro® fasteners, magnetic type fasteners, threaded type fasteners, sealable tongue and groove fasteners, snap fasteners, clip type fasteners, clasp type fasteners, ratchet type fasteners, a push-to-lock type connection method, a turn-to-lock type connection method, a slide-to-lock type connection method or any other suitable temporary connection method as one reasonably skilled in the art could envision to serve the same function. In further embodiments, one or more of the elements that comprise the device 200 may be coupled by being one of connected to and integrally formed with another element of the device 200.

Although the present invention has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention, are contemplated thereby, and are intended to be covered by the following claims.

What is claimed is:

1. A method for promoting eyelash growth, the method comprising:
a. applying an effective amount of a chitosan-biotin composition to eyelashes of a mammalian subject, wherein the chitosan-biotin composition comprises a chitosan biotin polymer comprising a chitosan to biotin ratio of between 1:1 to 1:10, and wherein the chitosan-biotin composition comprises between 0.001% to 0.1% of the chitosan biotin polymer.

2. The method of claim 1, wherein the chitosan-biotin composition comprises between 0.001% to 0.05% of the chitosan biotin polymer.

3. The method of claim 1, wherein the chitosan-biotin composition comprises between 0.002% to 0.01% of the chitosan biotin polymer.

4. The method of claim 1, wherein the chitosan to biotin ratio of the chitosan biotin polymer is between 1:1 to 1:5.

5. The method of claim 1, wherein the chitosan to biotin ratio of the chitosan biotin polymer is between 1:1 to 1:3.

6. The method of claim 1, further comprising applying the chitosan-biotin composition to a human eyelash line.

7. The method of claim 6, further comprising applying the chitosan-biotin composition to the human eyelash line at least once per day.

8. The method of claim 1, wherein the chitosan-biotin composition is a topical composition in a form selected from the group consisting of suspensions, oils, creams, ointments, gels, and lotions.

9. The method of claim 1, wherein the chitosan-biotin composition is a topical composition further comprising a carrier, wherein the carrier is selected from the group consisting of castor oil, Medium-Chain Triglyceride oil (MCT) oil, Glycerin, and Panthenol.

10. The method of claim 1, wherein the chitosan-biotin composition is a topical composition further comprising a carrier, wherein the carrier is selected from the group consisting of water, alcohols, aloe vera gel, allantoin, glycerin, vitamin A and E oils, mineral oil, propylene glycol, dimethyl isosorbide, polypropylene glycol, 2 myristyl propionate, and combinations thereof.

11. A method for promoting eyelash growth, the method comprising:
a. applying an effective amount of a chitosan-biotin composition to an eyelash line and eyelashes of a human subject, wherein the chitosan-biotin composition comprises a chitosan biotin polymer comprising a chitosan to biotin ratio of between 1:1 to 1:5, and wherein the chitosan-biotin composition comprises between 0.002% to 0.05% of the chitosan biotin polymer.

12. The method of claim 11, wherein the chitosan-biotin composition comprises between 0.002% to 0.01% of the chitosan biotin polymer.

13. The method of claim 11, wherein the chitosan-biotin composition comprises between 0.005% to 0.007% of the chitosan biotin polymer.

14. The method of claim 11, wherein the chitosan to biotin ratio of the chitosan biotin polymer is between 1:1 to 1:4.

15. The method of claim 11, wherein the chitosan to biotin ratio of the chitosan biotin polymer is between 1:1 to 1:3.

16. The method of claim 11, wherein the chitosan to biotin ratio of the chitosan biotin polymer is between 1:1 to 1:3, and wherein the chitosan-biotin composition comprises between 0.005% to 0.009% of the chitosan biotin polymer.

17. The method of claim 11, further comprising applying the chitosan-biotin composition to the eyelashes and the eyelash line of the human subject at least once per day.

18. The method of claim 11, wherein the chitosan-biotin composition is a topical composition in a form selected from the group consisting of suspensions, oils, creams, ointments, gels, and lotions.

19. The method of claim 11, wherein the chitosan-biotin composition is a topical composition further comprising a carrier, wherein the carrier is selected from the group consisting of castor oil, Medium-Chain Triglyceride oil (MCT) oil, Glycerin, and Panthenol.

20. The method of claim 11, wherein the chitosan-biotin composition is a topical composition further comprising a carrier, wherein the carrier is selected from the group consisting of water, alcohols, aloe vera gel, allantoin, glycerin, vitamin A and E oils, mineral oil, propylene glycol, dimethyl isosorbide, polypropylene glycol, 2 myristyl propionate, and combinations thereof.
